(19) United States
(12) Patent Application Publication
(10) Pub. No.: US 2024/0165219 A1
(43) Pub. Date: May 23, 2024

(54) Application of an Immunogenic or Vaccine Composition Against COVID-19

(71) Applicant: EYE VACC, Angleur (BE)

(72) Inventors: Norbert Schrage, Aix-la-Chapelle (DE); Frank Holzer, Saarbrucken (DE); Joël Blomet, Paris (FR); Daniel Desmecht, Louveigne (BE)

(73) Assignee: EYE VACC, Angleur (BE)

(21) Appl. No.: 18/283,061

(22) PCT Filed: Mar. 22, 2022
(86) PCT No.: PCT/EP2022/057548

§ 371 Date: Sep. 20, 2023

(30) Foreign Application Priority Data
Mar. 22, 2021 (DE) 102021001467.7
Jun. 18, 2021 (EP) 21180476.0
Jan. 20, 2022 (EP) 22152573.6

(51) Int. Cl. A61K 39/215; A61K 9/00; A61K 39/00; A61P 31/14

(52) U.S. Cl. CPC A61K 39/215; A61K 9/0048; A61P 31/14; A61K 2039/541

(57) ABSTRACT

The present invention relates to an immunogenic or vaccine composition against SARS-CoV-2, for use thereof in treatment of Covid-19, characterized in that it is administered on the ocular mucosa and/or the urogenital mucosa.

In addition to the systemic immune system, our bodies contain a mucosa associated immune system (called MALT, Mucosa Associated Lymphoid Tissue). The mucosa locally contain their own immune system, for example in the digestive tract (GALT, Gut Associated Lymphoid Tissue), in the nose (NALT, Nasal Associated Lymphoid Tissue), or even in the eye (CALT, Conjunctiva Associated Lymphoid Tissue).

In fact, the sites of entry of pathogens into the body are generally near the mucosa of the eyes, nose, mouth or gastrointestinal tract. Our bodies thus contain a large number of cellular and biochemical defense mechanisms directly at these mucosa, which activate on contact with pathogens. Typically the immune system of the mucosa comprises epithelial cells, innate immune cells and dendritic cells which are the interface between the innate immunity and specific (acquired) immunity. The mucosa may therefore contain innate immunity cells, immunocompetent cells, memory cells and antibody-producing cells.

After having experienced a primary infection by a pathogen, the body develops a systemic (serum) immunity against this agent. Further, if the pathogen was in contact with the mucosa of the body, mucosal immunity is also developed, in addition to systemic immunity.

Vaccines which can develop mucosal immunity are already known. Those vaccines are administered essentially by nasal or oral route. They present the singular advantage of inducing a protective immune response, both in the mucosa and at the systemic level. They also target blocking crossing of the mucosa, gateway for most bacterial and viral pathogens. This is, for example, the case of the FluMist® flu vaccine which is administered by nasal spray, or even polio vaccines which are administered orally. The flu virus (influenza A virus) vaccination, by ocular route, in ferrets was also studied in the article Eyedrop Vaccination Induced Systemic and Mucosal Immunity against Influenza Virus in Ferrets de Sangchul Yoon, Eun-Do Kim, Min-Suk Song, Soo Jung Han, Tae Kwann Park, Kyoung Sub Choi, Young-Ki Choi, and Kyoung Yul Seo; PLoS One. 2016; 11(6): e0157634. Ocular route vaccination was also studied in mice by the team of Kyoung Yul Seo, Soo Jung Han, Hye-Ran Cha, Sang-Uk Seo, Joo-Hye Song, So-Hyang Chung, and Mi-Na Kweon (Eye Mucosa: An Efficient Vaccine Delivery Route for Inducing Protective Immunity; J. Immunol 2010; 185:3610-3619), even though mice are not natural hosts of the virus.

In the case of pathogens that colonize the mucosa and are highly contagious, the pathogen multiplies so rapidly after having colonized the mucosa that the infection of the mucosa and the deeper layers progresses faster than the organism which can not establish effective immunity for eliminating the pathogen. This is typically the case of the current infection by Severe Acute Respiratory Syndrome Corona Virus 2 (SARS-CoV-2). In fact, COVID-19 (or coronavirus disease 2019) mainly presents an infection and inflammation of the mucosa with a secondary severe general disease, as is also the case for the flu, herpes or) poliomyelitis.

The current COVID-19 pandemic led to unprecedented research, in particular in order to develop vaccines, and now several COVID-19 vaccines have already been authorized for vaccinating the global population. Examples of such vaccines are: (1) the Pfizer/BioNTech mRNA vaccine, generally called Bnt162b2 or Comirnaty®; (2) the Moderna mRNA vaccine generally called, Moderna mRNA-1273, Spikevax or Moderna COVID-19 Vaccine; (3) the nonreplicating viral vector vaccine from AstraZeneca, generally called Vaxzevria®, ChAdOx1-S or AstraZeneca COVID-19 Vaccine; (4) the nonreplicating viral vector vaccine from Janssen/Johnson & Johnson, generally called Ad26COV2.S, JMJ Vaccine or J & J COVID-19; (5) the nonreplicating viral vector vaccine from Gamaleya, generally called Sputnik V or Gam-COVID-Vac; (6) the inactivated whole anti-COVID-19 vaccine with adjuvant from Sinovac R&D, generally called CoronaVac; or else (7) the recombinant nanoparticle subunit vaccine with adjuvant (Matrix M) from Novavax, generally called Nuvaxovid or NVX-CoV2373.

All currently available COVID-19 vaccines are administered by intramuscular route. They therefore produce a good systemic immunity, with which to limit severe progression of the disease, but they do not serve to develop immunity in the mucosa. This immunity in the mucosa is however essential for the prevention of new infections, without which the risk of SARS-CoV-2 infection will remain high.

Further, the quantities of vaccine currently available are still too low, and possible side effects of these vaccines (such as rare cases of thrombosis) are also a source of concern for some people, who thus hesitate to get vaccinated.

There is therefore still a need for improving vaccination of the global population against COVID-19 as a way to get out of this pandemic crisis.

The present invention addresses this need with a new mode of vaccination against COVID-19 which allows an improvement of the immunity (double immunity—systemic and mucosal—in fact produces a better protection against the pathogen, and thus contributes to the development of collective immunity), while also reducing the risk of severe side effects associated with vaccination. The present invention thus serves to develop IgM type and then IgG type immunoglobulins, but especially secretory IgA type immunoglobulins (sIgA on the mucosa). The present invention also aims to provide a new mode for COVID-19 vaccination which is the most effective possible.

The present invention also aims to provide a new mode for COVID-19 vaccination which is simpler and quicker to implement, in order that way increase the vaccination tempo.

The present invention serves to develop mucosal immunity against SARS-CoV-2, by targeting the ocular mucosa and/or the urogenital mucosa. The present invention also aims to develop an immunity called sterilizing. In fact, an objective of the present invention is to target the mucosa in order to mostly develop IgA type immunoglobulins (monomer IgA, but also dimer IgA and secretory IgA on the mucosa), in particular for the purpose of blocking viral replication in the mucosa of the respiratory tract (which is possible by means of IgA type and not IgG type immunoglobulins).

The present invention thus relates to an immunogenic or vaccine composition against SARS-CoV-2, for use thereof in prevention and/or treatment of Covid-19, characterized in that it is administered on the ocular mucosa and/or the urogenital mucosa.

The interest in administering such a composition is in establishing both an immunity in the mucosa and a serum immunity against SARS-CoV-2. This double immunity serves to better protect the organism against the virus and limits the risk of infections, without experiencing severe systemic side effects which may occur with some vaccines currently injected intramuscularly (in particular rare cases of thrombosis). In fact, in the case of the present invention, since the serum immunity is acquired indirectly via immunization of the mucosa, a thrombotic accident is therefore not expected.

According to the invention, the expression “immunogenic composition” and/or “vaccine composition” is understood to mean a composition which induces an immune response against SARS-CoV-2 after administration to the subject. A vaccine composition serves in particular to generate immunity, more specifically a protective and adaptive immune response against SARS-CoV-2. This immune response may be humoral and/or cellular. This immunity also aims to be immunizing.

According to the invention the term “SARS-CoV-2” is understood to mean the virus belonging to the Coronaviridae family, to the Betacoronavirus genus and to the Sarbecovirus subgenus. It is an enveloped virus with helicoidal capsid whose genome is made up of single-stranded RNA with about 30,000 nucleotides. The structure of the SARS-CoV-2 virion in particular includes a helicoidal capsid formed of N protein, a matrix formed of M protein and a lipid envelope in which at least two protein types are enclosed: the S (glyco)protein (spike) and the small envelope protein (E) (and potentially hemoagglutinin-esterase (HE)). The S protein and the variants are the main target of the immune response by production of neutralizing antibodies. This surface protein binds to the ACE2 receptor (which is expressed in many tissues), which allows the virus to penetrate the cells of the contaminated organism. The S protein contains two subunits, S1 and S2. S1 includes the receptor binding domain (RBD) which contains the receptor binding motif (RBM). The S2 subunit contains a fusion peptide which allows fusion between the cellular membrane of the cellular host (the contaminated organism) and the viral envelope.

More specifically, according to the invention the term “SARS-CoV-2” is understood to mean the virus for which the sequence was initially described in GenBank under the accession number MN908947, and also all variants of this virus, in particular the English variant, the South African variant, the Indian variant and the Brazilian/Japanese variant. Generally, that is understood to mean in particular the variants called Alpha (B.1.1.7), Beta (B.1.351), Gamma (P.1), Delta (B.1.617.2) et Omicron (B.1.1.529). That is also understood to mean the variants B.1.640, B.1.427, B.1.429, B.1.616, B.1.525, P.3, B.1.617.2, B.1.620, B.1.617.3, B.1.214.2, A.23.1, A.27, A.28, C.16, B.1.351, B.1.526, B.1.526.1, B.1.526.2, P.2, B.1.1.519, AV.1, AT.1, C.36, B.1.621, C.37, AY.4.2, B.1.1.318, B.1.617.2, C.1.2 or BA.2. Information about the SARS-CoV-2 sequences, mutations and sequences of variants can be found, for example, at the following links: https://www.ncbi.nlm.nih.gov/sars-cov-2/; https://www.ulc.gov/coronavirus/2019-ncov/cases-updates/variant-surveillance/variant-info.html; https://www.eulc.europa.eu/en/covid-19/variants-concern; or even https://www.gisaid.org/.

According to the invention, the expression “the prevention of Covid-19” is understood to mean prophylaxis. The administration of the immunogenic or vaccine composition according to the invention serves in particular to block or reduce the risk of developing Covid-19, and/or reduce as applicable the risk of developing forms of the disease referred to as serious (meaning with severe symptoms such as respiratory difficulties, and/or requiring hospitalization, very often in the intensive care unit). The administration of the immunogenic or vaccine composition according to the invention could also block or reduce the risk of transmission of the virus, typically from one person to another. According to a specific embodiment, the immunogenic or vaccine composition for the prevention of Covid-19 according to the invention is administered to a subject who has not been contaminated, preferably not been infected, by SARS-CoV-2.

According to the invention, the expression “the treatment of Covid-19” is understood to mean therapy. The administration of the immunogenic or vaccine composition according to the invention may in fact be considered in order to stimulate the natural defenses of the body, even though the person is a minima already contaminated by the virus. According to a specific embodiment, the immunogenic or vaccine composition for the treatment of Covid-19 according to the invention is administered to a subject who has been contaminated, and/or infected, by SARS-CoV-2.

Preferably, the present invention relates to an immunogenic or vaccine composition against SARS-CoV-2, for use thereof in prevention of Covid-19, characterized in that it is administered on the ocular mucosa and/or the urogenital mucosa.

According to the invention, the expression “the ocular mucosa” is understood to mean the conjunctiva and the cornea. The conjunctiva is the transparent mucosal membrane which covers the inner surface of the upper and lower eyelids and which covers the anterior surface of the eyeball. More precisely, the ocular mucosa is understood therefore to mean both the tarsal conjunctiva and the bulbar conjunctiva, along with the conjunctival fornix.

According to the invention, the expression “urogenital mucosa” is understood to mean the mucosa of the urinary and/or genital system, both the male and the female system. More precisely, the urogenital mucosa is understood therefore to mean the urogenital tract.

The targeting of the ocular mucosa and/or the urogenital mucosa serves to target mucosal which (i) in themselves only participate weakly in the spread of the pathogen, but (ii) which at the same time have a very high immunocompetence, which serves to immunize the body very rapidly, in particular before an infection of the respiratory pathways.

One of the inventors' nonlimiting hypothesis is in fact that infection of the eye by SARS-CoV-2 leads to rapid formation of immunity, allowing the body to gain time such that when the contamination and/or the infection progresses to the nose, the body is already immunized. This progression of the contamination and/or infection from the eye to the nose could be explained by the presence of the nasolacrimal canal which connects the nose to the eyes. An infection by the nasolacrimal canal has already been described in the case of keratitis caused by APC viruses (Adeno-Pharyngo Conjonctivales, Adenovirus of Keratitis epidemica type). The typical progression of this disease starts with an infection of the conjunctiva by droplets and is manifested by severe conjunctivitis followed by pharyngitis. This could also explain why subjects, in particular medical personnel, have developed systemic immunity against SARS-CoV-2 after having developed conjunctivitis positive to SARS-CoV-2.

Preferably, said immunogenic or vaccine composition is administered on the ocular mucosa.

According to another even more preferred embodiment, the invention thus relates to an immunogenic or vaccine composition against SARS-CoV-2, for use thereof in prevention of a Covid-19, characterized in that it is administered on the ocular mucosa.

According to the invention, said immunogenic or vaccine composition may be administered in only one or in both eyes, preferably in both eyes. Unlike intramuscular administration of the vaccine which requires an increased organization and medical effort (e.g. preparation of syringes, requiring availability of resuscitation equipment in case of severe reaction to the vaccination, etc.), the injection on ocular mucosa, typically by collyrium, is simpler and quicker to implement. In fact, the severe risks are reduced (for example risks of thrombosis as indicated above), as are the severe allergic risks which are principally seen as a tingling sensation in the eyes, tearing or even red eyes. Vaccination by collyrium further serves to vaccinate much more quickly and at a larger scale.

According to an embodiment, said immunogenic or vaccine composition may be administered in one or both eyes as a first dose of the vaccination schedule, or else as a booster. According to an embodiment, said immunogenic or vaccine composition is administered on the ocular mucosa and/or the urogenital mucosa before or after at least one administration of an immunogenic or vaccine composition intramuscularly. Preferably, said immunogenic or vaccine composition is administered on the ocular mucosa before or after at least one intramuscular administration of an immunogenic or vaccine composition. Preferably, said vaccine composition is administered on the ocular mucosa before or after at least one intramuscular administration of a vaccine composition. Typically, that means that the subject is vaccinated intramuscularly a first-time and then booster(s) are administered on the mucosa, in particular the ocular mucosa. This may also mean that the subject is vaccinated a first time intramuscularly, that one or more boosters are also done intramuscularly, and then one or more additional boosters are done by administration on the mucosa, notably ocular. The administration of an immunogenic or vaccine composition on the ocular mucosa and/or the urogenital mucosa after one or more intramuscular injections of a vaccine may be attractive if new boosters are desirable for targeting specific variants of the SARS-CoV-2 virus. Generally, administration on the ocular mucosa and/or the urogenital mucosa of immunogenic or vaccine composition according to the invention after at least one intramuscular administration of an immunogenic or vaccine composition has the objective of potentializing the acquired immunity after the first intramuscular administration(s). This also means that the first administration of the immunogenic or vaccine composition may be done on the ocular mucosa and/or the urogenital mucosa, and that the booster(s) may be done on the same mucosa or else by intramuscular route.

According to an embodiment, said immunogenic or vaccine composition comprises one or more substances serving to cause an immune response against SARS-CoV-2. Typically, said immunogenic or vaccine composition according to the invention comprises one or more antigens for SARS-CoV-2. Preferably, said antigen(s) are specific for SARS-CoV-2.

According to an embodiment, the immune or vaccine composition serves to cause an immune response against SARS-CoV-2, once it comprises at least one antigen for SARS-CoV-2, a microorganism (for example a virus or bacteria) which can produce at least one antigen for SARS-CoV-2, or else it comprises the genetic material necessary for the expression of at least one antigen for SARS-CoV-2 (typically an mRNA). In the first case, the antigen will be in contact with the mucosa right after administration and in the second and third case a period of latency could be expected, the time after administration of the composition during which the antigen is produced. Preferably, said microorganism is not pathogenic per se, the only immune response that it may cause is that related to the SARS-CoV-2 antigen.

According to a specific embodiment, the substances/antigens serving to cause an immune response against SARS-CoV-2 are selected from: (i) an inactivated SARS-CoV-2 virus, (ii) an attenuated SARS-CoV-2 virus (iii) a modified SARS-CoV-2 virus, preferably inactivated or attenuated, expressing or able to express one or more SARS-CoV-2 antigens (such as a SARS-CoV-2 viral protein), (iv) a genetically modified microorganism (e.g. bacteria or virus, preferably inactivated or attenuated) expressing or able to express one or more SARS-CoV-2 antigens (such as a SARS-CoV-2 viral protein), (v) a messenger ribonucleic acid (mRNA) coding for one or more SARS-CoV-2 antigens (such as a SARS-CoV-2 viral protein), (vi) deoxyribonucleic acid (DNA) coding for one or several SARS-CoV-2 antigens (such as a SARS-CoV-2 viral protein), (vii) a SARS-CoV-2 viral protein or one or more fragments thereof, or (viii) a fusion protein comprising a SARS-CoV-2 viral protein, or one or more fragments thereof . . . . The substances/antigens serving to cause an immune response against SARS-CoV-2 may also be (ix) a SARS-CoV-2 virus, meaning a living virus not inactivated or not attenuated, or even (x) a recombinant cell (for example a dendritic cell) expressing or able to express one or more SARS-CoV-2 antigens, (xi) a DNA plasmid coding for one or more SARS-CoV-2 antigens (such as a SARS-CoV-2 viral protein), or (xii) pseudo-viral particles comprising one or more SARS-CoV-2 antigens.

According to a specific embodiment, the substances/antigen serving to cause an immune response against SARS-CoV-2 are a genetically modified microorganism (in particular an adenovirus) expressing or able to express one or more SARS-CoV-2 antigens (such as a SARS-CoV-2 viral protein) or a SARS-CoV-2 virus, preferably a genetically modified microorganism (in particular an adenovirus) expressing or able to express one or more SARS-CoV-2 antigens (such as a viral vector). A viral protein fragment preferably contains the immunodominant epitope(s) or biosimilars thereof. According to an embodiment, the viral protein fragment is an immunogenic fragment. Said viral protein fragment may be recombinant.

According to a specific embodiment, the substances/antigens serving to cause an immune response against SARS-CoV-2 are selected from: (i) an inactivated SARS-CoV-2 virus, (ii) an inactivated SARS-CoV-2 virus (iii) a modified SARS-CoV-2 virus, preferably inactivated or attenuated, expressing or able to express one or more antigens (such as a viral protein for the SARS-CoV-2 virus) SARS-CoV-2 virus (iv) a genetically modified microorganism (e.g. bacteria or virus, preferably inactivated or attenuated) expressing or able to express one or more SARS-CoV-2 antigens such as a SARS-CoV-2 viral protein), (v) a messenger ribonucleic acid (mRNA) coding for one or more SARS-CoV-2 antigens (such as a SARS-CoV-2 viral protein), (vi) deoxyribonucleic acid (DNA) coding for one or several SARS-CoV-2 antigens (such as a SARS-CoV-2 viral protein), (vii) a SARS-CoV-2 virus.

The composition according to the invention may comprise one or more substances (antigens) serving to cause an immune reaction against SARS-CoV-2. According to an embodiment, the various substances (antigens) may target different parts of the same virus (for example different epitopes on viral proteins), different strains of the same virus, variants of the same virus and even several different viruses (combined vaccine). The immunogenic or vaccine composition according to the invention may thus be monovalent or polyvalent. A monovalent immunogenic or vaccine composition protects against a single pathogen, whereas a polyvalent immunogenic or vaccine composition protects against several pathogens.

According to an embodiment, the expression “a SARS-CoV-2 viral protein” is understood more specifically to mean structural proteins and accessory proteins of the virus, preferably structural proteins. In that way, according to an embodiment, said viral protein is selected from: the S, HE, M, N or E proteins or the ORF proteins, more specifically, ORF1a, ORF1b, ORF3, ORF6, ORF7a, ORF7b, ORF8a, ORF8b. Preferably, said viral protein is selected from the S, HE, M, N or E, proteins, more specifically the S, HE, M or E, and even more specifically the S protein. An immunogenic fragment of the S protein is more specifically the receptor binding domain (RBD), the 51 subunit or the cleavage region between the 51 subunit and the S2 subunit. According to the invention, viral proteins is understood to mean native proteins of the virus (proteins of the virus such as found in nature), proteins that are mutated or variant compared to the native proteins or synthetic proteins (modified, mutated or not).

The immunogenic or vaccine composition according to the invention is administered to a subject. According to an embodiment, said immunogenic or vaccine composition is used in the prevention and/or treatment of Covid-19 in humans. According to an embodiment, said immunogenic or vaccine composition is used in the prevention and/or treatment of Covid-19 in a child or an adult, in particular an adult. According to the invention, an adult is understood to be a person starting at 16 years old, preferably a person starting from 18 years old. Even more preferably, the adult has an age over or equal to 18 years old and up to 55 years old. According to an embodiment, said immunogenic or vaccine composition may be administered to a subject at least 12 years old. According to an embodiment, said immunogenic or vaccine composition may be administered to a subject at least 5 years old. According to an embodiment, said immunogenic or vaccine composition is also used in the prevention and/or treatment of Covid-19 in a baby and in a person over 55 years old.

According to another embodiment, veterinary applications of said immunogenic or vaccine composition according to the invention are conceivable. In such a case, the subject is then an animal.

According to an embodiment, said immunogenic or vaccine composition is administered to a subject in an immunologically effective quantity. Such a quantity may be determined by the practitioner. The expression “an immunologically effective quantity” is understood to mean a sufficient quantity for being effective at prevention and/or therapy, in particular in a subject needing such prevention or such treatment. As an example, the dosage of the vaccine composition may be between 0.005 mL and 0.05 mL for each of one to several epiocular drops or subconjunctival injections spaced at least one week apart, preferably between 2 to 12 weeks, for the AstraZeneca nonreplicating viral vector vaccine, the Sputnik V vaccine, the Johnson & Johnson vaccine, the Sinovac inactivated COVID-19 vaccine, or even the Nuvaxovid vaccine from Novavax.

According to an embodiment, said immunogenic or vaccine composition is administered to a subject who has not been contaminated, or even infected, by SARS-CoV-2 virus, responsible for the infection or else to a subject who has already been contaminated, even infected, by SARS-CoV-2 virus (that the person may have developed symptoms (mild or severe) or have been asymptomatic).

According to an embodiment, said immunogenic or vaccine composition is administered once or several times, preferably one, two or three times, on the ocular mucosa and/or the urogenital mucosa. According to a specific embodiment, said immunogenic or vaccine composition is administered at least twice on the ocular mucosa and/or the urogenital mucosa (in other words two doses of immunogenic or vaccine composition are administered). According to a specific embodiment, when the immunogenic or vaccine composition is administered several times, it is the same mucosa which is targeted: for example two or three administrations on the ocular mucosa, or two or three administrations on the urogenital mucosa. Preferably, at least one week separates the first and second administration, more specifically a time included between 4 to 12 weeks. The expression “between 1 to 12 weeks” means (days from 7 to 84 days) 1, 2, 3, 4, 5, 6, 7, 8, 9, 10, 11 or 12 weeks, or between 7 to 84 days.

According to an embodiment, said immunogenic or vaccine composition is administered as a drop, lyophilizate or other dry form. According to an embodiment, said immunogenic or vaccine composition may also be administered as dry, powder, gel, nanoparticle or pomade composition form. Typically, when the administration is in drop form, that means that said immunogenic or vaccine composition is a liquid formulation, and when the administration is in lyophilizate or dried composition form, said immunogenic or vaccine composition is in powder form. Said powder may be applied directly to the mucosa, or else be applied on filter paper, polymer, gel, nanoparticles or pomade or any other appropriate substances support which will be placed in contact with the ocular mucosa and/or urogenital mucosa. The immunogenic or vaccine composition will thus be transferred from the filter paper, polymer, gel or any other appropriate substance or support to the ocular mucosa and/or urogenital mucosa. Said dried composition may be obtained after freezing of the immunogenic or vaccine composition according to the invention and then dried with any appropriate technique (except for freeze-drying which serves to obtain a lyophilizate).

According to an embodiment, said immunogenic or vaccine composition is administered by using an applicator, for example which makes instilling drops (of eye-drop aid type) or powder or lyophilisate easier. The applicator may also be an ampoule, syringe, filter paper (with for example fluid or lyophilizate), a dropper bottle, a single dose applicator, a vaginal spiral or sponge type applicator, or even a fabric, a hygienic tampon comprising a modified outer surface (i.e. said surface comprising one or more substances serving to cause an immune reaction against SARS-CoV-2 such as a protein, a viral vector, etc.), or a condom comprising a modified external surface, etc.

According to an embodiment, the immunogenic or vaccine composition comprises or is made up of one or more substances (antigens) serving to cause an immune reaction against SARS-CoV-2. According to an embodiment, it may be considered that the immunogenic composition according to the invention is included in a vaccine composition (a vaccine). The term “vaccine” or “vaccine against Covid-19” may also be used in place of the expression “vaccine composition.” As an example, the immunogenic or vaccine composition according to the invention comprises (1) the Pfizer/BioNTech mRNA vaccine, generally called Bnt162b2 or Comirnaty®; (2) the Moderna mRNA vaccine generally called, Moderna mRNA-1273 or Moderna COVID-19 Vaccine; (3) the nonreplicating viral vector vaccine from AstraZeneca, generally called Vaxzevria®, ChAdOx1-S or AstraZeneca COVID-19 Vaccine, or its equivalent Covishield®; (4) the nonreplicating viral vector vaccine from Janssen, generally called Ad26COV2.S, JMJ Vaccine or J & J COVID-19; (5) the nonreplicating viral vector vaccine from Gamaleya, generally called Sputnik V or Gam-COVID-Vac or even its light version called Sputnik light; (6) the inactivated whole anti-COVID-19 vaccine with adjuvant from Sinovac R&D, generally called CoronaVac; (7) the recombinant nanoparticle subunit vaccine with adjuvant (Matrix M) from Novavax, generally called Nuvaxovid, Corovax or NVX-CoV2373; (8) the viral-like particle vaccine with the SARS-CoV-2 spike protein from Medicago, generally called CoviFenz®; (9) the protein subunit vaccine from Baylor College of Medicine/BioE Limited, generally called Corbevax® or BioE COVID-19; (10) the DNA plasmid based vaccine from Cadila Healthcare, generally called ZyCoV-D; or even (11) the inactivated virus vaccine from Bharat Biotech, generally called Covaxin®.

According to a preferred embodiment, sad vaccine composition comprises an adjuvant and/or an excipient.

According to an embodiment, said immunogenic or vaccine composition comprises a pharmaceutically acceptable vehicle.

The adjuvant and/or the excipient and/or the pharmaceutically acceptable vehicle are those conventionally used. For example, the pharmaceutically acceptable vehicle may be a solvent or solution serving to dilute the composition before administration by ocular route or on the urogenital mucosa.

According to an embodiment, an immunogenic or vaccine composition according to the invention may further comprise one or more additional substances. Preferably, said additional substance is selected from:
a preservative,
a marker, such as a colorant,
a surfactant, and
an additive.

According to an embodiment, the immunogenic or vaccine composition according to the invention is characterized in that it is administered after the administration of the first marker (such as a colorant) and before the administration of the second marker (such as a colorant). The immunogenic or vaccine composition according to the invention is thus administered between two markers. The administration of these two markers, preferably different, serves to verify that the immunogenic or vaccine composition according to the invention was correctly administered to the mucosa, in particular ocular mucosa.

As an example, a preservative may be an antimicrobial preservative whose purpose is to block microbial contamination of the immunogenic or vaccine composition. According to the invention, the preservative is a preservative compatible with the mucosa.

According to the invention, a “marker” is understood to mean any measurable or indicating substance which may be administered in an immunogenic or vaccine composition. It therefore involves a pharmaceutically acceptable marker. The marker also serves to verify the quantity administered and/or the administration site, where this is done in order to assure a safe and effective application of the immunogenic or vaccine composition.

According to a specific embodiment, the marker serves to quantify and/or visualize the application of said immunogenic or vaccine composition on the ocular mucosa and/or the urogenital mucosa. More specifically, quantification is understood to mean the measurement of the quantity of immunogenic or vaccine composition administered. According to an embodiment, the marker is an indicating substance such as a colorant. As an example, fluorescein or indocyanine type colorants may be used. This marker in particular serves to track the application of the immunogenic or vaccine composition on the ocular and/or urogenital mucosa. The coloring may also serve to track the dissolution of the immunogenic or vaccine composition on the ocular and/or urogenital mucosa. Visualizing the marker (such as the coloring) on the ocular and or urogenital mucosa serves to confirm that the immunogenic or vaccine composition was properly applied on the ocular and/or urogenital mucosa, even the quantity of immunogenic or vaccine composition administered.

In order to obtain an elevated antigenicity and therefore a greater immunological response, substances, for example surfactants, may be added to the immunogenic or vaccine composition in order to extend the exposure time on the ocular and/or urogenital mucosa, and possibly to allow calculating the exposure time of said composition on the mucosa.

Other substances, such as additives, may be added for conferring advantageous properties to the immunogenic or vaccine composition, for example a substance improving the penetration of the immunogenic or vaccine composition in the mucosa or a substance serving to extend the contact time between the mucosa and the composition (for example hyaluronic acid, one or more polymers for forming a hydrogel (e.g. carbomers), cellulose derivatives, etc.

The present invention also relates to a method for prevention and/or treatment of COVID-19 in a subject comprising the administration of an immunogenic or vaccine composition against SARS-CoV-2 on the ocular mucosa and/or the urogenital mucosa. More specifically, the present invention relates to a method for inducing a protective immune response against SARS-CoV-2, comprising the administration of an immunogenic or vaccine composition on the ocular mucosa and/or the urogenital mucosa.

The present invention also relates to the use of an immunogenic or vaccine composition against SARS-CoV-2, for the preparation of a medication intended to prevent and/or treat Covid-19, and where said medication is intended to be administered on the ocular mucosa and/or the urogenital mucosa.

### FIGURES

FIG. 1 shows the results for group 1 of hamsters.

FIG. 2 shows the results for group 2 of hamsters.

FIG. 3 shows the PENH whole body plethysmographic data for all groups before viral exposure (12 hours).

FIG. 4 shows the PENH whole body plethysmographic data for all groups three days after exposure to the SARS-CoV-2 virus.

FIG. 5 shows the PENH whole body plethysmographic data for all groups five days after exposure to the SARS-CoV-2 virus.

FIG. 6 shows the PENH whole body plethysmographic data for all groups seven days after exposure to the SARS-CoV-2 virus.

FIG. 7 shows the PENH whole body plethysmographic data for all groups 12 days after exposure to the SARS-CoV-2 virus.

FIG. 8 shows the PENH whole body plethysmographic data for all groups 14 days after exposure to the SARS-CoV-2 virus.

FIG. 9 shows the maximum dilution which always inhibits the SARS-CoV-2 infection in 50% of the cases (NT50) in the serum of the various hamster groups.

FIG. 10 shows the average of the weight of the hamsters in the four groups tested in Example 5.

### EXAMPLES

### Example 1: Examples of Liquid Compositions

Table 1 below summarizes the compositions according to the invention which may be used.

TABLE 1
Liquid compositions

| | | Viral | Number and type of |
|---|---|---|---|
| | Antigen or vaccine used | dose/concentration | applications |
| Composition 1 | The AstraZeneca nonreplicating | 0.001 × 10² to 10 × 10⁸ virus | One or more |
| | viral vector vaccine, generally | per mL, for example | applications, preferably |
| | called Vaxzevria ® or | 0.1 × 10² to 10 × 10⁸ virus | three |
| | AstraZeneca COVID-19 vaccine | per mL | Application on one or |
| | | For example, doses of | both eyes |
| | | 0.0001 to 0.1 mL or 20 μL | |
| | | may be administered | |
| Composition 2 | The Gamaleya nonreplicating | 0.001 × 10² to 10 × 10⁸ virus | One or more |
| | viral vector vaccine, generally | per mL, for example | applications, preferably |
| | called Sputnik V or Gam-COVID- | 0.1 × 10² to 10 × 10⁸ virus | three |
| | Vac | per mL | Application on one or |
| | | For example, doses of | both eyes |
| | | 0.01 to 0.1 mL or 20 μL | |
| | | may be administered | |
| Composition 3 | SARS-CoV-2 viral protein (S | 1 × 10⁻¹² g/mL to 1 g/mL | One or more |
| | protein type) | For example, doses of | applications, preferably |
| | | 0.0001 to 0.1 mL or 20 μL | three |
| | | may be administered | Application on one or |
| | | | both eyes |
| Composition 4 | Genetically modified non- | / | One or more |
| | pathogenic bacteria for producing | | applications, preferably |
| | a SARS-CoV-2 viral protein | | three |
| | | | Application on one or |
| | | | both eyes |

### Example 2: Examples of Freeze-Dried Compositions

Table 2 below summarizes the compositions according to the invention which may be used.

TABLE 2
Freeze-dried compositions

| | Antigen or | Number and type of |
|---|---|---|
| | vaccine used | applications |
| Composition 5 | Freeze-dried viral | One or more applications, |
| | protein, for | preferably three |
| | example the S protein | Application on one or both eyes |
| Composition 6 | Freeze-dried | One or more applications, |
| | virus (SARS-CoV-2, | preferably three |
| | inactivated or | Application on one or both eyes |
| | attenuated virus) | |

### Example 3: Immunization of Hamsters Against SARS-CoV-2 by Ocular Route

The hamsters have an ACE2 receptor. They may thus be contaminated by SARS-CoV-2 and become ill.

1. Comparison of Two Groups of Hamsters after Two Injections of Virus

Two groups of hamsters were studied.

In group 1, n=7 hamsters were contaminated with SARS-CoV-2 by nasal injection of a SARS-CoV-2 virus (B.1.214 strain) of 3×10⁶ and in group 2, n=7 hamsters were contaminated by means of a collyrium containing 3×10⁶ SARS-CoV-2 virus (B.1.214 strain).

During the observation, the hamsters from group 1 became ill, recognizable by a significant weight loss, whereas the hamsters from group 2 did not become ill.

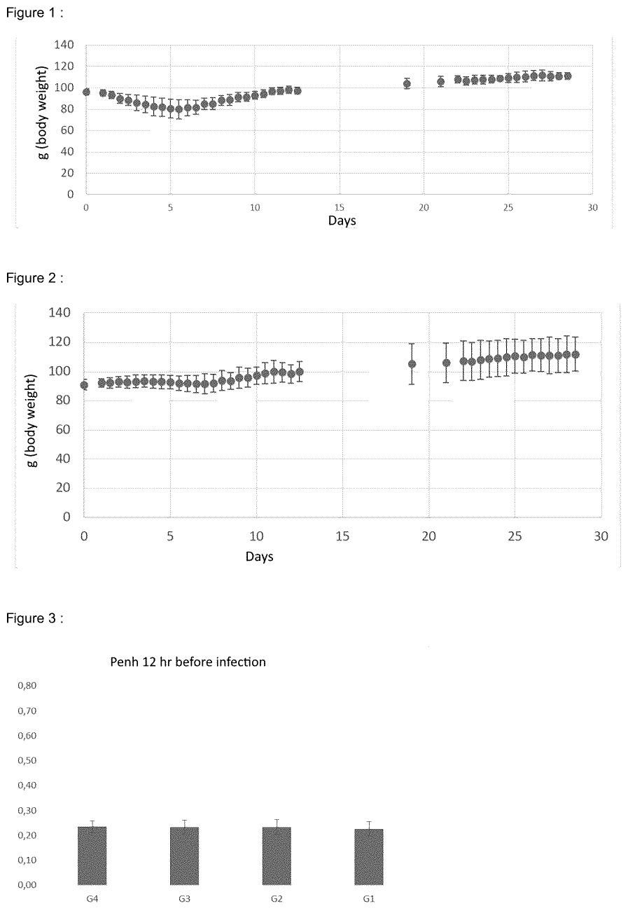

In fact, in FIG. 1 it can be seen that the hamsters from group 1 started to lose weight from day 2 with a maximum loss on day 5 and normalization on day 12; this indicates that the hamsters fell ill following SARS-CoV-2 contamination/infection by nasal injection. In contrast, in FIG. 2 it can be seen that the hamsters from group 2 do not lose weight after inoculation with SARS-CoV-2 by ocular route. This indicates that the animals remained in good health.

After 14 days, the two groups were again exposed to a nasal injection containing a SARS-CoV-2 viral dose of 3×10⁶.

In both groups, the hamsters continued to gain weight (see FIGS. 1 and 2). More specifically, at day 21, the animals from group 1 did not fall ill again after inhalation of the second SARS-CoV-2 viral dose. This means that they have become immune because of the illness developed during the first 10 days of the experiment.

Also, at day 21, the hamsters from group 2 did not fall ill after inhalation of the second SARS-CoV-2 viral dose. This means that the hamsters became immunized because of the first application of SARS-CoV-2 by ocular route.

The hamsters from group 2 thus acquired immunity with the epi-ocular application, which, unlike group 1, was acquired without developing a serious general illness.

2. Study of Hamsters after an Injection of Virus by Ocular Route

Further, the study of n=10 other hamsters which received a single application by ocular route of a composition containing a viral dose of 3×10⁶ SARS-CoV-2 showed that they developed a high production of neutralizing antibodies against the virus (at least 1:640 in the serum). These results mean that by epi-ocular application of the virus, the hamsters did not develop disease and acquired an immunity against SARS-CoV-2 which is detectable in the serum.

3. Plethsymographic Analysis

The pulmonary function of groups 1 and 2 mentioned above (see point 1) were measured using four single-camera, whole-body plethsymographs from EMKA Technologies (model PLT-UNR-RT-3). Each plethysmography chamber was equipped with the same wired pneumotachograph and the same differential pressure transducer (EMKA Technologies, model USB_DP_T). An opening in each chamber allowed continuous extraction of stale air at a constant rate (500 mL/minute) and the continuous replacement by fresh air coming from the room by means of a dedicated pump (model VENT_4_PLT). The measurement campaign per animal tested lasted about 20 minutes. The following procedures were then used for recording the flow rate signal generated in the plethsymographic chambers by a calm and alert respiration. The flow rate was systematically calibrated before and after the experiment. If a deviation of more than 5% was detected, the collected data were rejected and the measurement campaign was started over.

The raw-data curves were acquired by sampling the signals at 2 kHz. The regularity of the respiratory pattern was first evaluated manually by checking the constancy of the flow rate peaks. On the basis of this criterion, except for a few cases of coughing and grooming, most hamsters breathe regularly between the 5^{th} and 20^{th} minutes spent in the chamber. Unlike laboratory mice, hamsters are animals which can go from an alert state to a deep sleep state and back in a few seconds. The respiratory pattern was very different between those two states, it is crucial to select episodes of regular respiration during the watchful state. Also, the exploratory behavior these animals is much more intense than mice or rats and is accompanied by a specific respiratory pattern consisting of intense sniffing. The selection of a single episode of regular respiration is therefore insufficient. For this reason, the selection of episodes to analyze must be systematically done manually by experienced staff, and the resulting flow rate curves are then processed using IOX2 software (IIOX_1 PULMO_4a from EMKA Technologies). Thoracic-abdominal flow rate curves are then analyzed for determining the inspiratory time (TI) and the expiratory time (TE), the maximum inspiratory flow rate (PIF), and the maximum expiratory flow rate (PEF) and the current volume (TV). The latter was systematically measured two times per cycle, once during the inspiratory part (TI) and once during the expiratory part (TE) of the respiratory cycle. Two other parameters were calculated: the time needed to exhale the first 64% of the TV, called relaxation time (RT), and the bronchoconstriction index (Penh) proposed by Hamelmann et al., using the formula Penh=[(TE/RT)−1]*(PEF/PIF) (Hamelmann E, Schwarze J, Takeda K, Oshiba A, Larsen G L, Irvin C G, Gelfand E W. Noninvasive measurement of airway responsiveness in allergic mice using barometric plethysmography. Am J Respir Crit Care Med. 1997 September; 156(3 Pt 1):766-75. doi: 10.1164/ajrccm.156.3.9606031. PMID: 9309991).

Finally, the respiratory frequency [RR=60/(TI+TE)], the minute volume (MV=RR*TV), the mean inspiratory flow rate (MIF=TV/TI), the mean expiratory flow rate (MEF=TV/TE) and the cyclic ratio [% TI=TI/(TI+TE)] were also calculated from the parameters measured above and the body weight (BW). Based on the resulting quantitative values, the median value was systematically determined and used in order to calculate a representative average value for the hamster and for the day on which the analysis was done.

Results:

The Penh is a dimensionless, composite value which may be used for screening for pulmonary distress in laboratory animals.

The results are shown in the form of four groups, as indicated in Table 3 below.

TABLE 3
Description of the hamsters analyzed

| Groups | Type of infection |
|---|---|
| G1 | Nasal injection of 3 × 10⁶ of a SARS-CoV-2 virus |
| G2 | Nasal injection of 3 × 10⁶ of a SARS-CoV-2 virus, and then |
| | new nasal injection of 3 × 10⁶ of a SARS-CoV-2 virus 14 days |
| | later |
| G3 | Epiocular application of 3 × 10⁶ of the SARS-CoV-2 virus |
| G4 | Epiocular application of 3 × 10⁶ of a SARS-CoV-2 virus, and |
| | then new nasal injection of 3 × 10⁶ of a SARS-CoV-2 virus 14 |
| | days later |

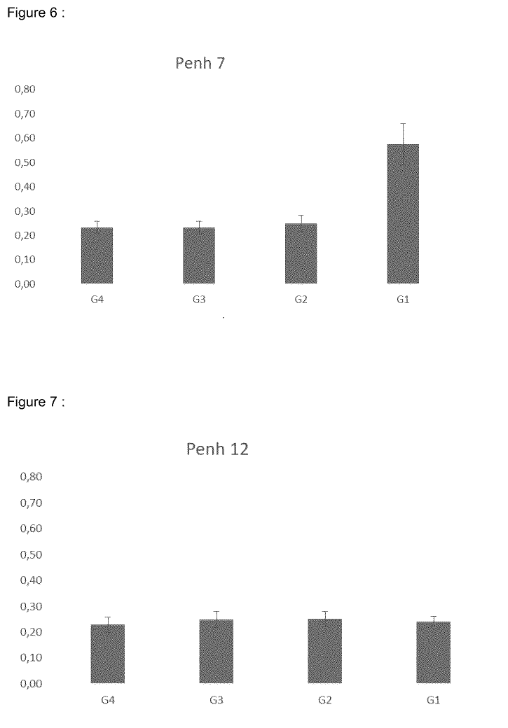

The results are presented in FIGS. 3 to 8.

Before infection, all the animals had a similar ventilation pattern, whatever the group (FIG. 3). After infection with the SARS-CoV-2 virus, two different patterns were observed.

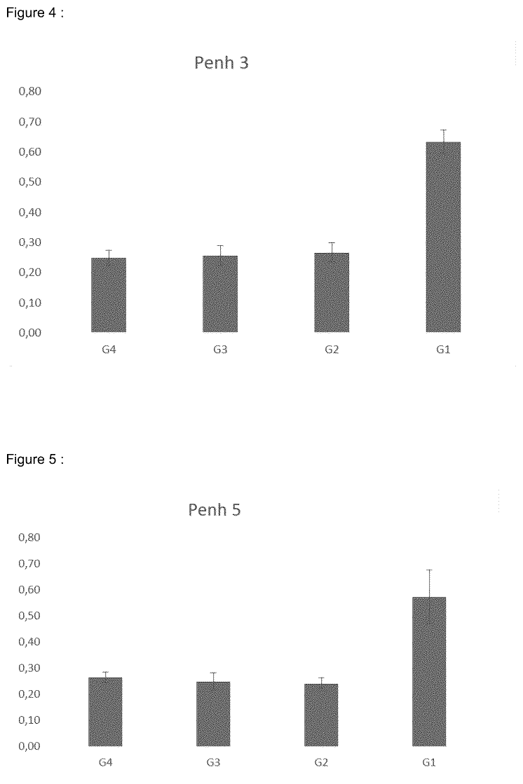

First, the animals from groups G2, G3 and G4 had a pattern very similar to that observed before infection, without significant difference, unlike the group G1 (FIGS. 4 to 6).

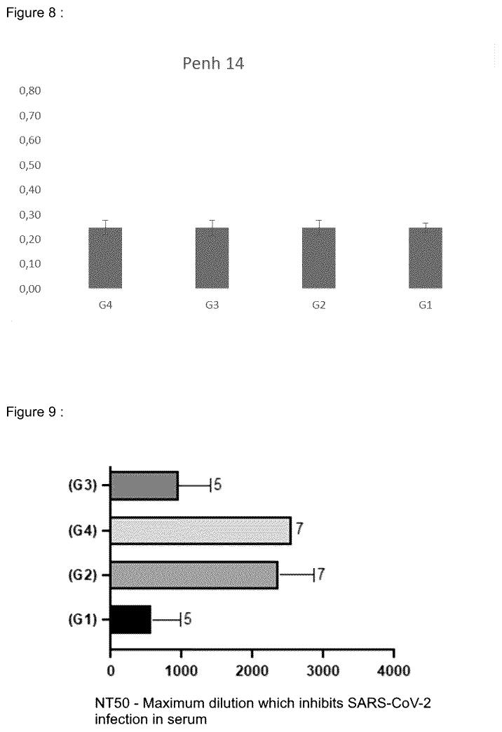

A return to normal was observed Monday 12 (post-infection) and hamsters from group G1 (FIGS. 7 and 8).

4. Evaluation of Neutralizing Antibodies in the Serum

A whole blood sample from the hamsters was collected in a cryotube, coagulated at ambient temperature for 1 to 2 hours and stored at 4° C. for 24 hours. The blood was then centrifuged, and the serum was placed in cryotubes and decomplemented at 56° C. for 45 minutes. The decomplemented serums were then stored at −20° C. until their use for determining the neutralizing antibody titer.

The virus stock was titrated in logarithmic dilutions in series to obtain a tissue culture infectious dose at 50% (TCID50) on 96-well culture plates. The plates were observed daily with an inverted optical microscope for five days in order to evaluate the presence of cytopathic effect (ECP) and the final titer was calculated according to the Reed & Muench method.

A serum sample was stored for each of the hamsters for quantification of the neutralizing antibody titers. The virus neutralization test was done with the BetaCov/Belgium/SartTilman/2020/1 strain of SARS-CoV-2 in 96-well plates containing confluent Vero E6 cells (ATCC CRL-1586).

Nine dilutions of each heat inactivated serum (40 minutes at 56° C.) were used (1:10 to 1:1280—corresponding to final dilutions for the test 1:20 to 1:2580). The dilutions were done in three exemplars in DMEM/FBS on 96-well culture plates.

The serums (50 μL/well) were mixed with an identical volume (vol/vol) of a solution containing 100 TCID50 (Tissue Culture Infection Dose 50) of SARS-CoV-2 virus.

The serum-virus mixture was then incubated at 37° C. for one hour in a humidified atmosphere with 5% CO2.

After incubation, 100 μL of a suspension of Vero cells were added so as to deposit 20,000 cells in each well. The plates were then incubated again for five days. For each serum, the process was repeated twice. After five days, the ECP was evaluated under optical microscope. The serum dilutions showing an ECP were considered as non-neutralizing (negative), whereas those not showing any ECP were considered as positive/neutralizing.

The serum virus neutralization titer of the was reported as the highest serum dilution which neutralizes the ECP and 50% of the wells (NT50). For all the serums having NT50>1:320, a second process was done by using higher dilutions (up to 1:20480).

Positive controls (NT50=1:160, from the Belgian National Reference Center for Human Microbiology) and negative controls (saline solution) were inserted in each plate.

The results are presented in FIG. 9.

### Example 4: Study of the Propagation of SARS-CoV-2 During Infection of the Nasal Cavity

The Inventors analyzed the propagation of the virus in hamsters who were contaminated with SARS-CoV-2 via a nasal injection, such as described in Example 3.

The Inventors thus observed that in the first 48 hours following entry of the virus in the nose, the infection progressed slowly with an infection of the nasal cavity only in the mucosa, with formation of aerosols. These aerosols can then infect the bronchus and the alveoli, in particular during inhalation, and contaminate other people during exhalation.

### Example 5: Vaccination by Means of Different Immunogenic or Vaccine Compositions

Four new groups of hamsters were studied.

In group 1, 20 μL of vaccine with Janssen/Johnson & Johnson nonreplicating viral vector (generally called Ad26COV2.S), taken directly from the vial, without any modification, were administered in the left eye of n=8 hamsters on D=1. After 14 days (D=14), 20 μL of the same vaccine, taken directly from the vial, without any modification, were again administered in the left eye of n=8 hamsters.

In group 2, 50 μL of vaccine with Janssen/Johnson & Johnson nonreplicating viral vector (generally called Ad26COV2.S), taken directly from the vial, without any modification, were administered intramuscularly in both hips of n=8 hamsters on D=1. After 14 days (D=14), 50 μL of the same vaccine, directly taken from the vial, without any modification, were again administered intramuscularly in both hips of n=8 hamsters.

In group 3, 20 μL of PBS (phosphate buffered saline) was administered in the left eye of n=8 hamsters on day D=1. After 14 days (D=14), 20 μL of PBS (phosphate buffered saline) was again administered in the left eye of n=8 hamsters.

In group 4, 20 μL of a solution containing 3×10⁶ of a SARS-CoV-2 virus (Wuhan-like variant of the SARS-CoV-2 virus), (an attenuated virus, not inactivated virus) were administered in both eyes of n=8 hamsters at 14 days (D=14) of the experiment.

After 14 additional days (D=28 since the beginning of the experiment), the four groups were contaminated intranasally with 200 μL of a solution containing 6×10⁶ TCID50 (Tissue Culture Infection Dose Fifty) of the SARS-CoV-2 virus (Wuhan-like variant of the SARS-CoV-2 virus).

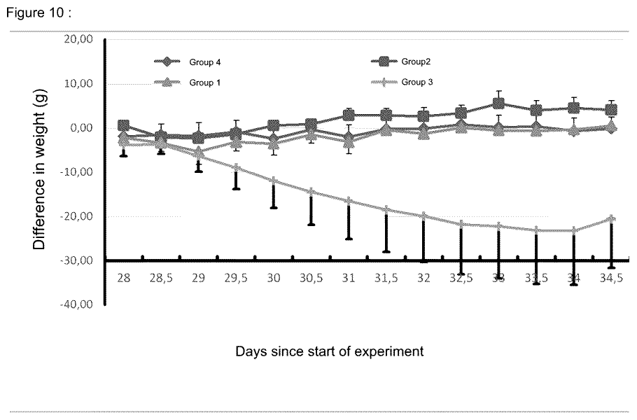

The weight loss of the animals (i.e. an indicator that the hamster was ill) was analyzed for each of the four groups. The results are presented in FIG. 10. The curves show the average weight of the hamsters in the four groups.

The results show the hamsters from groups 1, 2 and 4 are protected and do not fail ill, unlike the hamsters from group 3.

### Example 6: Ocular Vaccination in Humans

Vaccination by ocular route is tested in humans.

Two people were previously vaccinated intramuscularly (two injections of a COVID-19 vaccine in the deltoid muscle of the left arm) received 20 of a new dose of a COVID-19 vaccine administered on the ocular mucosa of the right eye, more than six months after the intramuscular injection.

The assay of the IgG in the serum and the IgA in the oral and pharyngeal mucosa was then done, by collection of blood and an oral and pharyngeal wash with 10 mL of 0.9% sodium chloride for two minutes on the day of administration of the new vaccine dose on the ocular mucosa, and then three weeks later. The results are presented in FIG. 4 below.

TABLE 4
IgG, IgM and IgA levels in the subjects

| | | IgM levels in | |
|---|---|---|---|
| | IgG levels in serum | serum | IgA levels in mucosa |
| | (BAU/mL) | (BAU/mL) | (BAU/mL) |
| Subject 1: Assay on the day of | 89.3 | 0 | 0.3 |
| administration of the vaccine | | | |
| dose on the ocular mucosa | | | |
| Subject 1: Assay three weeks | 160.8 | 0 | 12.4 |
| after administration of the | | | |
| vaccine dose on the ocular | | | |
| mucosa | | | |
| Subject 2: Assay on the day of | 109.4 | 0 | 0.2 |
| administration of the vaccine | | | |
| dose on the ocular mucosa | | | |
| Subject 2: Assay three weeks | 127 | 0 | 17.3 |
| after administration of the | | | |
| vaccine dose on the ocular | | | |
| mucosa | | | |

1. A method for the prevention and/or the treatment of Covid-19, wherein an immunogenic or vaccine composition against SARS-CoV-2 is administered on the ocular mucosa and/or the urogenital mucosa.

2. The method according to claim 1, wherein said composition is administered on the ocular mucosa.

3. The method according to claim 1, wherein said composition comprises one or more substances serving to cause an immune response against SARS-CoV-2.

4. The method according to claim 3, wherein the substances serving to cause an immune response against SARS-CoV-2 is selected from a SARS-CoV-2 virus, an inactivated SARS-CoV-2 virus, an attenuated SARS-CoV-2 virus, a modified SARS-CoV-2 virus expressing or able to express one or more SARS-CoV-2 antigens, a genetically modified microorganism expressing or able to express one or more SARS-CoV-2 antigens, a messenger ribonucleic acid (mRNA) coding for one or more SARS-CoV-2 antigens, a deoxyribonucleic acid (DNA) coding for one or several SARS-CoV-2 antigens, a SARS-CoV-2 viral protein or one or more fragments thereof, a fusion protein comprising a SARS-CoV-2 viral protein, or one or more fragments thereof, a recombinant cell expressing or able to express one or more SARS-CoV-2 antigens, a DNA plasmid coding for one or more SARS-CoV-2 antigens, or pseudo-viral particles.

5. The method according to claim 4, wherein said viral protein is selected from: the S, HE, M, N or E proteins or the ORF proteins.

6. The method according to claim 4, wherein said viral protein is the S protein or a fragment thereof.

7. The method according to claim 1, wherein said composition is administered in the form of a drop, lyophilizate, dried composition, powder, gel, nanoparticle or pomade.

8. The method according to claim 1, wherein said composition is administered by using an applicator.

9. The method according to claim 1, wherein said composition is administered in animals or humans.

10. The method according to claim 1, wherein said composition further comprises one or more additional substances.

11. The method according to claim 10, wherein the additional substance is selected from:
a preservative,
a marker, in particular an indicating substance, such as a colorant,
a surfactant, and
an additive.

12. The method according to claim 1, wherein said composition is administered after the administration of a first marker and before the administration of a second marker.

13. The method according to claim 1, wherein the administration on the ocular mucosa and/or the urogenital mucosa is done before or after at least one intramuscular administration of an immunogenic or vaccine composition.

14. The method according to claim 4, wherein said SARS-CoV-2 antigen is a SARS-CoV-2 viral protein.

15. The method according to claim 6, wherein said viral protein or a fragment thereof is the receptor binding domain (RBD), the S1 subunit or the cleavage region between the S1 subunit and the S2 subunit.
